(19) United States
(12) Patent Application Publication
(10) Pub. No.: US 2024/0006022 A1
(43) Pub. Date: Jan. 4, 2024

(54) METHODS AND SYSTEMS FOR DETECTING INSERTIONS AND DELETIONS

(71) Applicant: Guardant Health, Inc., Palo Alto, CA (US)

(72) Inventors: Marcin Sikora, Redwood City, CA (US); Mohammad R Mokhtari, Redwood City, CA (US); Darya Chudova, Redwood City, CA (US)

(21) Appl. No.: 18/469,290

(22) Filed: Sep. 18, 2023

Related U.S. Application Data
(63) Continuation of application No. 16/539,815, filed on Aug. 13, 2019.
Application No. 16/539,815 is a continuation of application No. PCT/US2018/033553, filed on May 18, 2018.

(51) Int. Cl. G16B 20/20; G16B 30/10; G16B 25/20

(52) U.S. Cl. CPC G16B 20/20; G16B 30/10; G16B 25/20

(57) ABSTRACT

Methods and systems for improving callings of insertions and/or deletions by identifying genetic sequence reads having identical molecular barcodes and sequences among sequence reads from a nucleic acid sequencer, grouping the genetic reads into a family, and processing families comprising split reads to detect the insertion and/or deletion in a sample of polynucleotide molecules.

### CROSS-REFERENCE

This application is a Continuation of U.S. application Ser. No. 16/539,815, filed Aug. 13, 2019, which is a Continuation of International Application No. PCT/US2018/033553, filed on May 18, 2018 which claims the benefit of U.S. Provisional Application No. 62/509,003, filed on May 19, 2017; 62/509,699, filed on May 22, 2017; and 62/511,186, filed on May 25, 2017, wherein each application is incorporated herein by reference in its entirety.

### BACKGROUND

Genetic variants, such as insertions, deletions, substitutions, rearrangements and copy number variants may be correlated with diseases. Next-generation sequencing technologies or high-throughput sequencing can be employed to detect genetic variants. Identifying genetic variants accurately is critical for using the next-generation sequencing technologies in identifying the genetic variants associated with diseases.

Genetic variants such as insertions and deletions represent the second most frequent class of genetic variants in a human genome, after single nucleotide polymorphisms. The insertions and/or deletions also contribute to pathogenesis of diseases, gene expression and functionality.

### SUMMARY

In an aspect, the present disclosure provides a system, comprising: (a) a communication interface that receives, over a communication network, sequence reads generated by a nucleic acid sequencer; and (b) a computer in communication with the communication interface, wherein the computer comprises one or more computer processors and a computer readable medium comprising machine-executable code that, upon execution by the one or more computer processors, implements a method comprising: i. receiving, over the communication network, the genetic sequence reads generated by the nucleic acid sequencer; ii. processing the genetic sequence reads to generate processed sequence reads; iii. mapping the genetic sequence reads to a reference sequence; iv. grouping the processed sequence reads into families, each family comprising unique sequence reads originating from the same polynucleotide molecule in a sample; v. grouping at least a portion of the families into fusion clusters, each fusion cluster comprising split reads, wherein each split read comprises a first sub-sequence adjacent to a first breakpoint that maps to a first genetic locus and a second sub-sequence adjacent to a second breakpoint that maps to a second, distinct genetic locus, and wherein the first breakpoint and the second breakpoint form a breakpoint pair; and vi. calling a fusion cluster as comprising an insertion and/or deletion where: breakpoint pairs map to the same chromosome, distance between the first breakpoint and the second breakpoint in the breakpoint pair is less than a predetermined maximum distance on the reference sequence, and sub-sequences are in the same 5′-3′ orientation. In some embodiments, the system further comprises calling a fusion cluster as having a fusion in which at least one of the above-mentioned criteria in (vi) is not met. In some embodiments, the system further comprises generating an electronic report which provides an indication of the polynucleotide molecules comprising the insertion, deletion and/or fusion.

In some embodiments, the processed sequence reads with the same start-stop positions on the reference sequence are grouped into a family. In some embodiments, the genetic sequence reads comprises paired end sequence reads. In some embodiments, the paired end sequences with overlapping regions are merged to generate processed reads comprise merged reads. In some embodiments, the paired end reads with an overlapping region having at least 70% identity are merged. In some embodiments, the paired end reads with an overlapping region having at least 80% identity are merged. In some embodiments, the paired end reads with an overlapping region having at least 90% identity are merged. In some embodiments, the paired end reads with an overlap of at least 13 bases are merged. In some embodiments, the paired end reads with an overlap of at least 15 bases are merged. In some embodiments, the paired end reads with an overlap of at least 17 bases are merged. In some embodiments, the paired end reads with an overlap of at least 19 bases are merged.

In some embodiments, the paired end sequences with overlapping regions are merged to form merged reads, and wherein the merged sequence reads are further processed to generate processed reads comprising representative, merged unique reads. In some embodiments, the at least a portion of the families comprise a plurality of split reads. In some embodiments, the system further comprises generating a consensus sequence for each family comprising the plurality of split reads. In some embodiments, the split reads are consensus sequences generated from each family.

In some embodiments, the distance between the first breakpoints of the split reads within the fusion cluster is less than 10 nucleotides from each other and the distance between the second breakpoints of the split reads within the fusion cluster is less than 10 nucleotides from each other. In some embodiments, the split-read is a consensus sequence of a family.

In some embodiments, the predetermined maximum distance is less than 5,000 nucleotides. In some embodiments, the predetermined maximum distance is less than 3,500.

In some embodiments, the families further comprise the families further comprise processed reads: (a) having the same start position and the same compacted stop sequence, or (b) having the same stop position and the same compacted start sequence.

In some embodiments, the compacted start/stop sequence is generated by compacting the entirety of the unique sequence read to remove duplicate nucleotides in a homopolymer. In some embodiments, the homopolymers comprise a poly(dA) or a poly(dT). In some embodiments, the homopolymers comprise a poly(dG) or a poly(dC).

In some embodiments, the sample comprises cell-free DNA. In some embodiments, the reference sequence is a human reference sequence. In some embodiments, the nucleic acid sequencer is a next-generation sequencer. In some embodiments, the paired end sequence reads are assessed for quality to generate quality scores.

In some embodiments, the computer readable medium comprises a memory, a hard drive or a computer server. In some embodiments, the communication network comprises a telecommunication network, an internet, an extranet, or an intranet. In some embodiments, the communication network includes one or more computer servers capable of distributed computing. In some embodiments, the distributed computing is cloud computing.

In some embodiments, the communication network includes a storage device comprising the genetic sequence reads.

In some embodiments, the computer is located on a computer server that is remotely located from the nucleic acid sequencer.

In some embodiments, the system further comprises an electronic display in communication with the computer over a network, wherein the electronic display comprises a user interface for displaying results upon implementing (i)-(vi). In some embodiments, the user interface is a graphical user interface (GUI) or web-based user interface. In some embodiments, the electronic display is in a personal computer. In some embodiments, the electronic display is in an internet enabled computer. In some embodiments, the internet enabled computer is located at a location remote from the computer.

In another aspect, the present disclosure provides a computer-implemented method for detecting insertions and/or deletions in genetic sequence reads, comprising: (a) receiving, with a computer processor, genetic sequence reads of polynucleotide molecules generated from a nucleic acid sequencer; (b) processing, with the computer processor, the genetic sequence reads to generate processed sequence reads; (c) mapping, with the computer processor, the processed sequence reads to a reference sequence; (d) grouping, by the computer processor, the processed sequence reads into families, each family comprising unique sequence reads originating from the same polynucleotide molecule in a sample; (e) grouping, by the computer processor, at least a portion of the families into fusion clusters, each fusion cluster comprising split reads, wherein each split read comprises a first sub-sequence adjacent to a first breakpoint that maps to a first genetic locus and a second sub-sequence adjacent to a second breakpoint that maps to a second, distinct genetic locus, and wherein the first breakpoint and the second breakpoint form a breakpoint pair; (f) calling, by the computer processor, fusion clusters as comprising an insertion and/or deletion where: i. breakpoint pairs are located on the same chromosome of the reference sequence, ii. distance between the first breakpoint and the second breakpoint in the breakpoint pairs is less than a predetermined maximum distance on the reference sequence, and iii. sub-sequences are in the same 5′-3′ orientation. In some embodiments, the method further comprises: (g) calling, by the computer processor, fusion clusters as comprising a fusion in which at least one of the criteria in (f) is not met.

In some embodiments, the systems and methods disclosed herein comprise calling a fusion cluster a deletion if the first and second sub-sequences are in normal genomic order as compared to the reference sequence. In other embodiments, the systems and methods disclosed herein comprise calling a fusion cluster an insertion if the first and second sub-sequences are in reverse genomic order as compared to the reference sequence.

In some embodiments, the genetic sequence reads comprise sets of paired end sequence reads. In some embodiments, the processing comprises: i. merging the paired end sequence reads to form merged reads. In some embodiments, the processing further comprises: ii. grouping collections of merged reads having identical barcodes and the same internal sequence into unique sets; and iii. generating the processed sequence read for each unique set. In some embodiments, the paired end sequence reads with overlapping regions are merged to form the merged sequence reads. In some embodiments, the paired end sequence reads with an overlapping region having at least 60% identity are merged. In some embodiments, the paired end reads with an overlapping region having at least 70% identity are merged. In some embodiments, the paired end reads with an overlapping region having at least 80% identity are merged. In some embodiments, the paired end reads with an overlapping region having at least 90% identity are merged. In some embodiments, the paired end reads with an overlap of at least 13 bases are merged. In some embodiments, the paired end reads with an overlap of at least 15 bases are merged. In some embodiments, the paired end reads with an overlap of at least 17 bases are merged. In some embodiments, the paired end reads with an overlap of at least 19 bases are merged.

In some embodiments, the distances between the first breakpoints of the split reads within the fusion cluster is less than 10 nucleotides from each other and the distances between the second breakpoints of the split reads within the fusion cluster are less than 10 nucleotides from each other. In some embodiments, the predetermined maximum distance is less than 5,000 nucleotides. In some embodiments, the predetermined maximum distance is less than 3,000 nucleotides.

In some embodiments, the processed sequence reads are grouped into families based on having a same pair of molecular barcodes. In some embodiments, the processed sequence reads are grouped into families based on mapping to a same location on the reference sequence.

In some embodiments, the processed sequence reads in the families comprise sequence reads: (a) having a same start position and a same compacted stop sequence, or (b) having a same stop position and a same compacted start sequence. In some embodiments, the compacted start or stop sequence is generated by compacting a portion of the processed sequence read to remove duplicate nucleotides in a homopolymer. In some embodiments, the homopolymers comprise a poly(dA) or a poly(dT). In some embodiments, the homopolymers comprise a poly(dG) or a poly(dC).

In some embodiments, the families are grouped into fusion clusters based on split reads having breakpoints within a predetermined breakpoint distance of one another. In some embodiments, the predetermined breakpoint distance is less than 25 nucleotides. In some embodiments, the predetermined breakpoint distance is less than 10 nucleotides.

In some embodiments, the split reads are consensus sequences generated for each of the families comprising split reads. In some embodiments, the consensus sequences are grouped into fusion clusters based on split reads having breakpoints within a predetermined breakpoint distance of one another. In some embodiments, the predetermined breakpoint distance is less than 25 nucleotides. In some embodiments, the predetermined breakpoint distance is less than 10 nucleotides.

In some embodiments, the reference sequence is a human reference sequence. In some embodiments, the nucleic acid sequencer is a next-generation sequencer.

In some embodiments, the sample is a bodily fluid obtained from a subject. In some embodiments, the bodily fluid is selected from the group consisting of blood, plasma, serum, urine, saliva, mucosal excretions, sputum, stool, and tears. In some embodiments, the subject has cancer. In some embodiments, the sample comprises cell-free DNA molecules.

In some embodiments, the method further comprises generating in electronic format which provides an indication of polynucleotide molecules having the insertions and/or deletions and/or fusions. the method further comprises generating in electronic format which provides an indication of polynucleotide molecules having the insertions and/or deletions and/or fusions.

In another aspect, the present disclosure provides a method, comprising: (a) mapping genetic sequence reads of polynucleotide molecules to a reference sequence; (b) identifying genetic sequence reads comprising split reads, wherein each split read comprises a first sub-sequence adjacent to a first breakpoint that maps to a first genetic locus and a second sub-sequence adjacent to a second breakpoint that maps to a second, distinct genetic locus, and wherein the first breakpoint and the second breakpoint form a breakpoint pair; (b) grouping the split reads into families, each family comprising sequence reads originating from the same polynucleotide molecule in a sample; (d) generating, for each family, a consensus split read sequence; (e) grouping consensus split read sequences for each family into fusion clusters, wherein the consensus sequences within the fusion cluster have similar breakpoint pairs; (f) calling fusion clusters as comprising an insertion and/or deletion where: i. breakpoint pairs are located on the same chromosome of the reference sequence, ii. distance between the first breakpoint and the second breakpoint in the breakpoint pairs is less than a predetermined maximum distance on the reference sequence, and iii. sub-sequences are in the same 5′-3′ orientation. In some embodiments, the method further comprises: (g) calling fusion clusters as comprising a fusion in which at least one of the criteria in (f) is not met.

In some embodiments, the consensus sequences in each fusion cluster comprise split reads having first breakpoints that are within a first predetermined breakpoint distance between one another and second breakpoints that are within a second predetermined breakpoint distance between one another. In some embodiments, the first predetermined breakpoint distance is less than 25 nucleotides. In some embodiments, the predetermined distance is less than 10 nucleotides. In some embodiments, the second predetermined breakpoint distance is less than 25 nucleotides. In some embodiments, the second predetermined distance is less than 10 nucleotides.

In another aspect, the present disclosure provides a method, comprising: (a) mapping genetic sequence reads of polynucleotide molecules to a reference sequence; (b) grouping the genetic sequence reads into families, each family comprising unique sequence reads originating from the same polynucleotide molecule in a sample; (c) grouping unique sequence reads of families into fusion clusters, each fusion cluster comprising split reads, wherein each split read is characterized by sub-sequences: a first sub-sequence adjacent to a first breakpoint that maps to a first genetic locus and a second sub-sequence adjacent to a second breakpoint that maps to a second, distinct genetic locus, and wherein the first breakpoint and the second breakpoint form a breakpoint pair; (d) calling unique sequence reads of fusion clusters as comprising an insertion and/or deletion where: i. breakpoint pairs map to the same chromosome; ii. distance between the first breakpoint and the second breakpoint in the breakpoint pair is less than a predetermined maximum distance on the reference sequence; and iii. sub-sequences are in the same 5′-3′ orientation. In some embodiments, the method further comprises: (e) calling unique sequence reads of fusion clusters as comprising a fusion in which at least one of the criteria in (d) is not met. In some embodiments, the method further comprises generating in electronic format which provides an indication of polynucleotide molecules having the insertions and/or deletions and/or fusions. the method further comprises generating in electronic format which provides an indication of polynucleotide molecules having the insertions and/or deletions and/or fusions.

In another aspect, the present disclosure provides a computer-implemented method for detecting insertions and/or deletions and/or fusions, comprising: (a) aligning and merging, with a computer processor, paired end sequence reads collected from a nucleic acid sequencer to generate representative merged, unique reads from sets of paired end sequence reads, wherein each representative merged, unique read represents paired end sequence reads having the same molecular barcodes and sequences after merging of the paired end sequence reads; (b) mapping, with the processor, the representative merged, unique reads to a reference sequence; (c) grouping, with the processor, the representative merged, unique reads into families, each family comprising representative merged, unique reads originating from the same original tagged polynucleotide molecule, each family represented by a consensus sequence; (d) grouping, with the processor, consensus sequences of families into fusion clusters, each fusion cluster comprising consensus sequences from a family of split reads, wherein each split read is characterized by sub-sequences, wherein a first sub-sequence adjacent to a first breakpoint that maps to a first genetic locus and a second sub-sequence adjacent to a second breakpoint that maps to a second, distinct genetic locus, wherein the first breakpoint and the second breakpoint form a breakpoint pair, wherein consensus sequences in the fusion cluster comprise similar breakpoint pairs; (e) calling, with the processor, fusion clusters having an insertion and/or deletion in which: (i) breakpoint pairs map to the same chromosome, (ii) distance between breakpoint pairs is less than a predetermined maximum distance, and (iii) sub-sequences are in the same 5′-3′ orientation. In some embodiments, the method further comprises calling, by the processor, fusion clusters having a fusion in which at least one of the following criteria is not met: i. breakpoint pairs map to the same chromosome, ii. distance between breakpoint pairs is less than a predetermined maximum distance, and iii. sub-sequences are in the same 5′-3′ orientation.

In some embodiments, the computer-implemented method further comprises calculating, with the processor, sequencing quality of the paired end sequence reads to provide quality scores for the paired end sequence reads.

In another aspect, the present disclosure provides a method for treating a patient with cancer, comprising: (a) receiving data as to the presence or amount of a fusion cluster in the patient, wherein the data is obtained using any of the above-mentioned methods; and (b) subjecting the patient to different treatment regimens based on the presence or amount of the fusion cluster.

In some embodiments, the patient with the fusion cluster or presence of higher amounts of the fusion cluster receive a more stringent therapeutic regime than patients without the fusion cluster or with lower amounts of the fusion cluster. In some embodiments, the more stringent regime is characterized by a higher dose of a therapeutic agent than a dose of a therapeutic agent in a less stringent regime.

In some embodiments, the fusion cluster is called as a MET exon 14 skipping deletion. In some embodiments, the therapeutic agent is a MET inhibitor. In some embodiments, the MET inhibitor is selected from the group consisting of crizotinib, cabozantinib, capmatinib, tepotinib, and glesatinib. In some embodiments, the treatment regime comprises chemo-, radio-, or immunotherapy.

In some embodiments, the data indicates the presence of the fusion cluster in patients receiving a treatment for cancer, and the treatment is continued in such patients.

All methods described herein can be a computer implemented method.

All methods described herein can further comprise generating a report in electronic format which provides an indication of polynucleotide molecules having the insertions and/or deletions and/or fusions.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1 illustrates an embodiment of the disclosure showing a workflow for detecting genetic variants.

FIG. 2 illustrates an embodiment of the disclosure showing a procedure for generating representative merged reads.

FIG. 3 illustrates an embodiment of the disclosure showing a procedure for determining a fusion cluster.

FIG. 4 shows an example computer control system that is programmed or otherwise configured to implement methods provided herein.

### DETAILED DESCRIPTION

The present disclosure provides methods and systems for detecting genetic variants, such as insertions, deletions and fusions in a sample of polynucleotide molecules, such as a mixed sample of cell-free DNA. The methods and systems described herein can detect different genetic variants with improved sensitivity and specificity. For example, the methods described herein can detect large insertions and/or deletions and/or fusions, such as up to 1,000 base pairs.

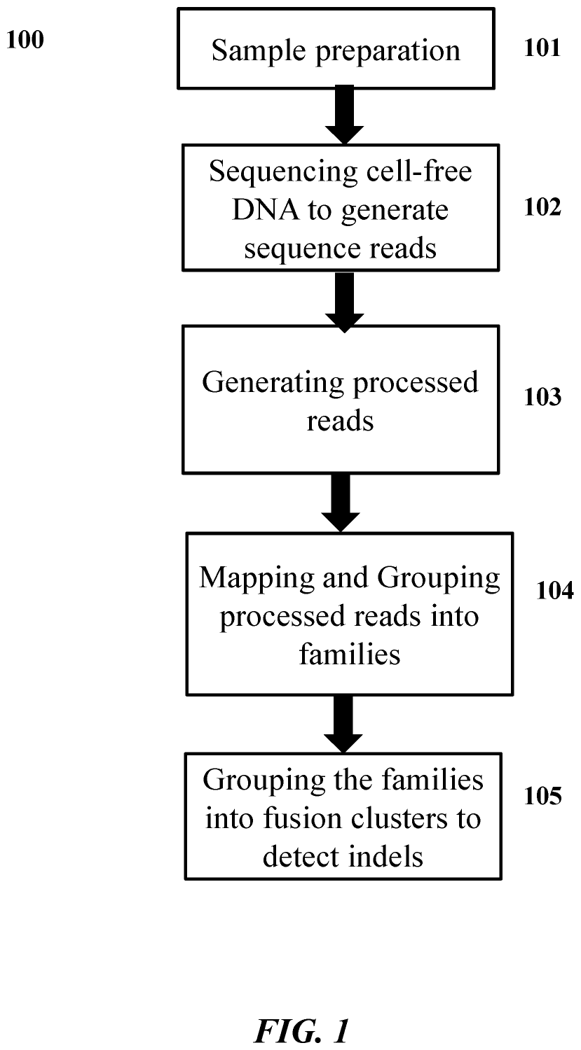

FIG. 1 illustrates an embodiment of the disclosure. In 101, a sample comprising polynucleotide molecules is prepared for sequencing. The polynucleotide molecules are tagged to generate tagged molecules. In 102, the tagged molecules are sequenced to generate genetic sequence reads. In 103, the genetic sequence reads are processed to generate processed reads. In 104, the processed reads are mapped to a reference sequence and grouped into families. In 105, the families are processed to detect genetic variants in the polynucleotide molecules.

In 101, a sample comprising polynucleotide molecules, such as a mixed sample of tumor derived and non-tumor derived polynucleotide molecules, is prepared for sequencing. Such preparation is dependent on the application and the sequencing platform used, for example a next-generation sequencing platform.

A sample can be any biological sample isolated from a subject. Samples can include body tissues, such as known or suspected solid tumors, whole blood, platelets, serum, plasma, stool, red blood cells, white blood cells or leukocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid (CSF), saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and/or single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid for analysis is plasma or serum containing cell-free nucleic acids, e.g., cell-free DNA (cfDNA).

The volume of body fluid can depend on the desired read depth for sequenced regions. Exemplary volumes are 0.4-40 ml, 5-20 ml, 10-20 ml. For examples, the volume can be 0.5 ml, 1 ml, 5 ml, 10 ml, 20 ml, 30 ml, or 40 ml. A volume of sampled plasma may be 5 to 20 ml.

The sample can comprise various amount of nucleic acid that contains genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free. A sample can comprise nucleic acids carrying mutations. For example, a sample can comprise DNA carrying germline mutations and/or somatic mutations. A sample can comprise DNA carrying cancer-associated mutations (e.g., cancer-associated somatic mutations). In some cases, nucleic acid can be found in an efferosome or an exosome.

Cell-free nucleic acids can be referred to all non-encapsulated nucleic acid sourced from a bodily fluid (e.g., blood, urine, CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA (ctDNA). Others are released from healthy cells. ctDNA can be non-encapsulated tumor-derived fragmented DNA. Cell-free fetal DNA (cffDNA) is fetal DNA circulating freely in the maternal blood stream.

Cell-free DNA is normally highly fragmented, with size distribution in the range of about 100-300 base pairs (bp) in length and so no additional fragmentation of it is required. For example, size of fetal and maternal cell-free DNA is approximately 162 bp while size of cell-free DNA that is tumor-derived can be approximately 166 bp. In instances where a sample may have long molecules of DNA, fragmentation is optional.

Cell-free nucleic acids can be isolated from bodily fluids through a partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, cell-free nucleic acids can be precipitated with an alcohol. Further clean up steps may be used such as silica based columns to remove contaminants or salts. Non-specific bulk carrier nucleic acids, for example, may be added throughout the reaction to optimize certain aspects of the procedure such as yield.

After such processing, samples can include various forms of nucleic acids including double-stranded DNA, single-stranded DNA and/or single-stranded RNA. Optionally, single stranded DNA and/or single stranded RNA can be converted to double stranded forms so they are included in subsequent processing and analysis.

Exemplary amounts of cell-free nucleic acids in a sample before amplification range from about 1 fg to about 1 ug, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Additional sequences, such as molecular barcodes and adapters may be attached to one or both ends of the polynucleotide molecules. Such additional sequences can be attached via primer hybridization or ligation reaction. Primer hybridization can include attachment of additional sequences through amplification reaction, such as polymerase chain reaction (PCR). Ligation reaction can include formation of a covalent bond between the additional sequences and the fragments of polynucleotide molecules. Ligation can be blunt end ligation or sticky end ligation. In some instances, the fragments of polynucleotide molecules may be modified prior to ligation reaction, such as introducing overhang nucleotides or amplifying the polynucleotide sequences.

The adapters may comprise oligonucleotide sequences complementary to a sequencing primer. For example, the adapters can include a sequencing primer binding site where a polymerase enzyme can bind and initiate polymerization for sequencing the polynucleotide molecules.

The adapters may comprise sequences enabling adapters to bind to a sequencing lane in the next-generation sequencing platform. For example, the adapters can include a flow cell attachment site for attaching to the sequencing lane in Illumina platform. The adapters can include sequence complementary to oligonucleotides attached to the sequencing lane in the next-generation sequencing platform. For example, the adapters can include complementary sequence that can hybridize with oligonucleotides attached to a flow cell of the sequencing lane in Illumina platform.

The adapters may comprise additional sequences such as a molecular barcode or an index or a tag. The molecular barcodes or indices or tags can be used to distinguish among the sequence reads derived from different samples. The molecular barcodes may be useful for multiplexing sequencing reaction with more than one sample. The molecular barcodes may be randomly or non-randomly tagged to either one end or both ends of the polynucleotide molecules. Where the polynucleotide molecules are tagged at both ends, the combination of barcodes may be referred to generically as an “identifier”. The molecular barcode may be attached between the adapter and a polynucleotide molecule. The molecular barcodes can be double stranded or single stranded. Preferably, an adapter is a Y-shaped adapter that includes a double stranded molecular barcode at its stem and/or a single stranded molecular barcode at the non-complementary end of the Y. In some embodiments, a sample is contacted with more distinct molecular barcodes than there are polynucleotide molecules in the sample. In other instances, a small number of distinct molecular barcodes is used to tag each of the polynucleotide molecules (e.g., less than the number of DNA molecules).

In certain embodiments, the molecular barcodes may be unique, such that a molecular barcode sequence is not shared by any other polynucleotide molecule in the sample. In this situation, the polynucleotide molecules are “uniquely tagged”. In some embodiments, the molecular barcodes may not be unique such that a molecular barcode sequence is shared by at least one other polynucleotide molecule in the sample. In this situation, the polynucleotide molecules in the sample are “non-uniquely tagged”. In an embodiment of non-unique tagging, the number of different barcodes is fewer than the total number of polynucleotide molecules in the sample.

The number of molecular barcodes used may be more than about 1, 2, 3, 4, 5, 6, 7, 8, 9, 10, 50, 100, 500, 1000, 5000, 10,000, 50,000, 100,000, 500,000, 1,000,000, 10,000,000, or 1,000,000,000. In some embodiments, the tagging format uses 5-10,000, 5-5,000, or 100 different molecular barcodes, ligated, optionally as part of adapters, to both ends of a target molecule. In some embodiments, the tagging format uses 20-50 different molecular barcodes, ligated, optionally as part of adapters, to both ends of a target molecule creating 20-50×barcodes, e.g., 400-2500 barcodes.

In another embodiment, the number of different barcodes or barcode combinations can be at least enough so that there is a 99.99% chance that the sequence reads generated from the polynucleotide molecules map to the same start/stop coordinates in a reference genome, or the sequence reads map at some point in their sequence (e.g., overlap a base position in a reference sequence) are uniquely tagged.

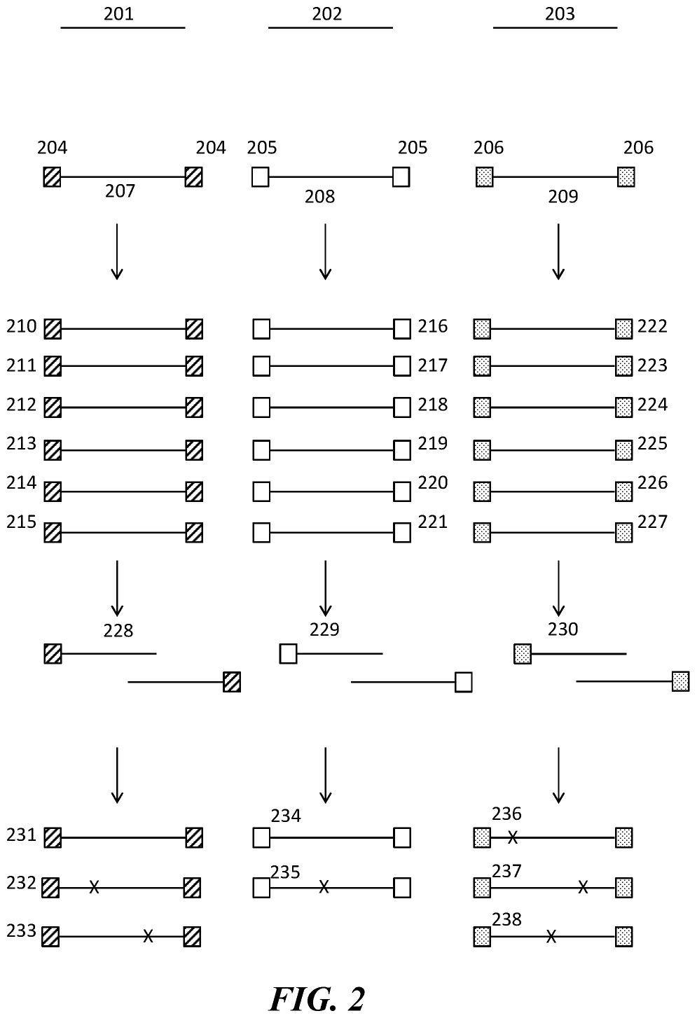

For example, as shown in FIG. 2, polynucleotide molecules 201, 202 and 203 are respectively tagged by 204, 205 and 206 molecular barcodes on both ends. The tagged molecules are then amplified to generated copies of the original polynucleotide molecule. For example, the tagged molecules 207, 208 and 209 are respectively amplified to generate 210-215, 216-221 and 222-227 amplicons.

In certain embodiments, the polynucleotides can be enriched prior to sequencing. Enrichment can be performed for specific target regions (“target sequences”) or nonspecifically. In some embodiments, targeted regions of interest may be enriched with capture probes (“baits”) selected for one or more bait set panels using a differential tiling and capture scheme. A differential tiling and capture scheme uses bait sets of different relative concentrations to differentially tile (e.g., at different “resolutions”) across genomic regions associated with baits, subject to a set of constraints (e.g., sequencer constraints such as sequencing load, utility of each bait, etc.), and capture them at a desired level for downstream sequencing. These targeted genomic regions of interest may include regions of a subject's genome or transcriptome. In some embodiments, biotin-labeled beads with probes to one or more regions of interest can be used to capture target sequences, optionally followed by amplification of those regions, to enrich for the regions of interest.

Sequence capture typically involves the use of oligonucleotide probes that hybridize to the target sequence. A probe set strategy can involve tiling the probes across a region of interest. Such probes can be, e.g., about 60 to 120 bases long. The set can have a depth of about 2×, 3×, 4×, 5×, 6×, 8×, 9×, 10×, 15×, 20×, 50×, or more. The effectiveness of sequence capture depends, in part, on the length of the sequence in the target molecule that is complementary (or nearly complementary) to the sequence of the probe.

In some embodiments, the methods of the disclosure comprise selectively enriching regions from the subject's genome or transcriptome prior to sequencing. In other embodiments, the methods of the disclosure comprise non-selectively enriching regions from the subject's genome or transcriptome prior to sequencing.

In certain embodiments, sample index sequences are introduced to the polynucleotides after enrichment. The sample index sequences may be introduced through PCR or ligated to the polynucleotides, optionally as part of adapters.

Referring back to FIG. 1, in 102, tagged polynucleotide molecules are sequenced. Sequencing is preferably performed using next-generation sequencing platforms, such as Illumina™, Ion Torrent™, Pacific Biosciences sequencing systems, or Oxford Nanopore sequencing technologies. Sequencing produces raw sequencing data comprising sequence reads that are long reads or short reads. Long reads can be more than 1 kilobases (kb) in lengths while short reads can be less than 1 kb in lengths.

Certain sequencing systems produce redundant reads for each original polynucleotide molecule, for example, by amplification of the polynucleotide molecule and subsequent sequencing of amplicons. Certain sequencing systems, such as Illumina, produce paired end sequence reads, that is, sequence reads from both ends of the molecule which pairs of reads may or may not overlap. Other sequencing systems can produce a single sequence read sequence of an entire polynucleotide molecule. In the sequencing systems that do not produce paired end reads, the step of merging reads can be eliminated and represented reads can be selected from the full-length reads.

The methods as shown in FIG. 1 can be implemented using a computer. For example, a computer-implemented method can be used for detecting insertions and/or deletions and/or fusions. The method may include an algorithm for calculating quality of paired end sequence reads collected from a sequencer with a computer processor. For example, quality scores for paired end sequence reads based on the quality of sequencing may be provided. The paired end sequence reads may further be aligned and merged to generate representative merged, processed reads from sets of paired end sequence reads. Each representative merged, processed read represents paired end sequence reads that have the same molecular barcodes and internal sequences.

The raw sequencing data comprising sets of paired end sequence reads can be provided in various file formats, such as FASTQ, VCF, CRAM or BAM. Files with the raw sequencing data may include sequence data for one strand or both strands, such as in paired-end reads. In one example, the raw sequencing data is provided in a FASTQ file for both strands i.e. sense and antisense strands generated from paired end sequencing procedure. The files may include additional symbols providing information about the quality of reads and may also provide a quality score. The raw sequencing data of each polynucleotide molecule may be saved on a local drive, in cloud or a server.

It is expected that in a collection of sequence reads, e.g. paired end reads, there will be a plurality of reads having the same sequence. This is particularly the case when original polynucleotide molecules are amplified, producing many copies, and the amplicons are sequenced. Accordingly, any particular sequence in a set of sequence reads can be considered a “unique sequence” for which there may be a plurality of copies in the set. Unique sequence reads can be selected from the sets of all sequences used in the mapping steps disclosed herein.

In 103, processed reads are generated from the genetic sequence reads from the sequencer. Processing may include any method that makes the analysis of the genetic sequence reads more efficient. For example, in some cases, processing may include merging paired end genetic sequence reads to form a merged read. In some cases, processing may include grouping collections of merged reads having identical barcodes and a substantially similar or the same internal sequence into unique sets and generating a representative merged read. In other cases, processing may include trimming the tags from the genetic sequence reads. 103 removes duplicate sequence reads and eliminates substantial computational analysis.

For example, as shown in FIG. 2, sets of paired end reads 228, 229 and 230 each comprise two mate pairs. The mate pairs are merged to form a merged read. The collections of the merged reads having the same barcodes and a substantially similar or the same internal sequence are grouped into unique sets. Then, a representative merged, unique read for each unique set is selected. For example, the representative merged, unique reads 231, 232 and 233 are generated for the paired end sequence reads for 201 after grouping the merged reads into unique sets based on, for example, the molecular barcodes and the internal sequence. Similarly, the representative merged, unique reads 234 and 235 are generated for the paired end sequence reads for 202. The representative merged, unique reads 236, 237 and 238 are generated for the paired end sequence reads for 203.

Alternatively, unique sequences (based on a combination of barcodes and internal sequence) are determined from among sets of paired end reads. Then, paired end reads are merged to generate representative merged, unique sequence reads.

A sense strand of a paired end sequence read is merged with an antisense strand of a paired end sequence read. For example, the paired end sequence reads are reoriented to be antiparallel and then merged to form a merged read or a mate pair. The mate pair or the merged read comprises the sense strand and the antisense strand having an overlapping region. The overlapping region may comprise at least about 1 base, 2 bases, 3 bases, 4 bases, 5 bases, 10 bases, 15 bases, 20 bases, bases, 30 bases, 35 bases, 40 bases, 45 bases, 50 bases, 55 bases, 60 bases, 65 bases, 70 bases, bases, 80 bases, 85 bases, 90 bases, 95 bases, or 100 bases. The identity of bases between the strands in an overlapping region can be at least about 5%, 10%, 15%, 20%, 25%, 30%, 35%, 40%, 45%, 50%, 55%, 60%, 65%, 70%, 75%, 80%, 85%, 90%, 95%, or more. In some cases, a given overlapping region can comprise at least 15 bases with at least about 90% identity between the strands. In other cases, the overlapping can comprise at least 19 bases with at least 90% identity between the strands. The overlapping region is represented by a strong peak when using sliding window analysis. For example, the overlapping region is slid to include a base on each end of the overlapping region and identity between the strands is computed until both strands completely overlap each other. The identity between the strands is computed as percentage of identity. The percentage of identity is directly proportional to the height of the peak. The merged reads or the mate pairs with a single strong peak are selected for further analysis.

Referring back to FIG. 1, in 103, both strands of the merged reads may be trimmed to remove at least a portion of the sequence at 3′ ends in the overlapped region. For example, half of the sequence in the overlapped region at 3′ ends can be removed to exclude bases with low sequence quality, molecular barcodes on 3′ ends, and any mismatches. This step is useful in reducing sequencing errors.

In 104, the processed reads, including merged reads or representative, merged reads (depending on the processing step) are aligned to a reference sequence using mapping tools, non-limiting examples of which may include Burrow's Wheeler Transform (BWA), Novoalign, Bowtie. The mapping tools generate an alignment file describing alignment parameters used, position of the representative merged, unique reads (such as coordinates) on to the reference sequence and a quality score of mapping. The alignment parameters, such as number of differences allowed between the sequencing read and the reference sequence, number of gaps allowed and gap opening penalty, number of gap extensions, and the like, may be defined by a user.

In one instance, BWA mapping tool with default alignment parameters is used to align the processed reads to a human reference genome, such as hg19. BWA tool provides an output file, a BAM file that includes alignment statistics. Alignment statistics may include coordinates of the reference sequence to which the processed reads align to. Alignment statistics may also provide a MapQ score to inform uniqueness of the processed reads when mapped to the reference sequence. The processed reads may then be sorted using the molecular barcodes and the coordinates on the reference sequence.

In some embodiments, the genetic sequence reads from the nucleic acid sequencer are not processed and may be aligned or mapped to the reference sequence.

The processed reads may be grouped into families. A family comprises reads originating from the same original tagged polynucleotide molecule. The processed reads also have the same mapping coordinates on the reference sequence. For example, the processed reads having a pair of molecular barcodes (e.g. Tag 1 and Tag 2) and an endogenous sequence that aligns to the same coordinates on the reference sequence (e.g. 1200-1500 on chromosome 1) may be grouped into a family. In some embodiments, each family may be represented by a consensus sequence (a “family consensus sequence”). The processed reads may be added to the family if the processed reads have the same molecular barcodes and at least one end position on the reference genome similar to the rest of reads in the family. For example, the processed reads may have the same molecular barcode and the same start position but stop positions may be within a predetermined nucleotide range. If the processed reads have a same compacted stop sequence upon compaction, the processed reads are grouped into the same family.

Similarly, the processed reads may have the same molecular barcode and the same stop position but start positions may be within a predetermined nucleotide range. If the processed reads have the same compacted start sequence upon compaction, the processed reads are grouped into the same family.

The processed reads can be compacted to remove duplicate nucleotides in a homopolymer. Duplicate nucleotides in a homopolymer can be removed within a predetermined range of less than 2 nucleotides, 3 nucleotides, 4 nucleotides, 5 nucleotides, 6 nucleotides, 7 nucleotides, 8 nucleotides, 9 nucleotides, 10 nucleotides, 20 nucleotides, 30 nucleotides, 40 nucleotides, or 50 nucleotides. In some cases, the predetermined range can be less than 10 nucleotides. In some cases, the predetermined range can be less than 7 nucleotides. In some cases, the predetermined range can be less than 5 nucleotides. In some cases, the predetermined range can be less than 3 nucleotides. In one instance, the predetermined range is 4 nucleotides. Upon compaction, if at least 7 nucleotides in the end sequence map to the same position on the reference sequence as the rest of the representative merged, unique reads, then the compacted reads are grouped into the same family. Compacting of the merged reads reduces the number of families produced due to sequencing errors, for example, at the ends of a sequence read.

In certain embodiments, one or more homopolymers may be present at the start sequence and/or the stop sequence. The one or more homopolymers may be present anywhere in the processed reads. In some embodiments, the homopolymers may comprise a poly(dA) or a poly(dT). In other embodiments, the homopolymers may comprise a poly(dG) or a poly(dC).

As an example, for two processed reads, if the start position of the first processed read is within the predetermined range, such as less than 5 nucleotides, of the start position of the second processed read and the first 7 bases of the compacted sequence of the first processed read is identical to the first 7 bases of the compacted sequence of the second processed read and the end positions of first processed read and second processed read are identical, then these reads can be grouped into the same family. Likewise, if the end position of the first processed read is within the predetermined range, such as less than 5 nucleotides, of the end position of the second processed read and the last 7 bases of the compacted sequence of the first processed read is identical to the last 7 bases of the compacted sequence of the second processed read and the start positions of first processed read and second processed read are identical, then these reads can be grouped into the same family.

The families with the processed reads can be aligned to a reference sequence to identify split reads that do not contiguously align to the reference sequence. For example, each split read can be characterized by sub-sequences. A first sub-sequence maps to a first genetic locus while a second sub-sequence maps to a second genetic locus. The first genetic locus is distinct from the second genetic locus. The first sub-sequence maps to a first genetic locus adjacent a first breakpoint and the second sub-sequence maps to a second genetic locus adjacent a second breakpoint. The first breakpoint and the second breakpoint can form a breakpoint pair.

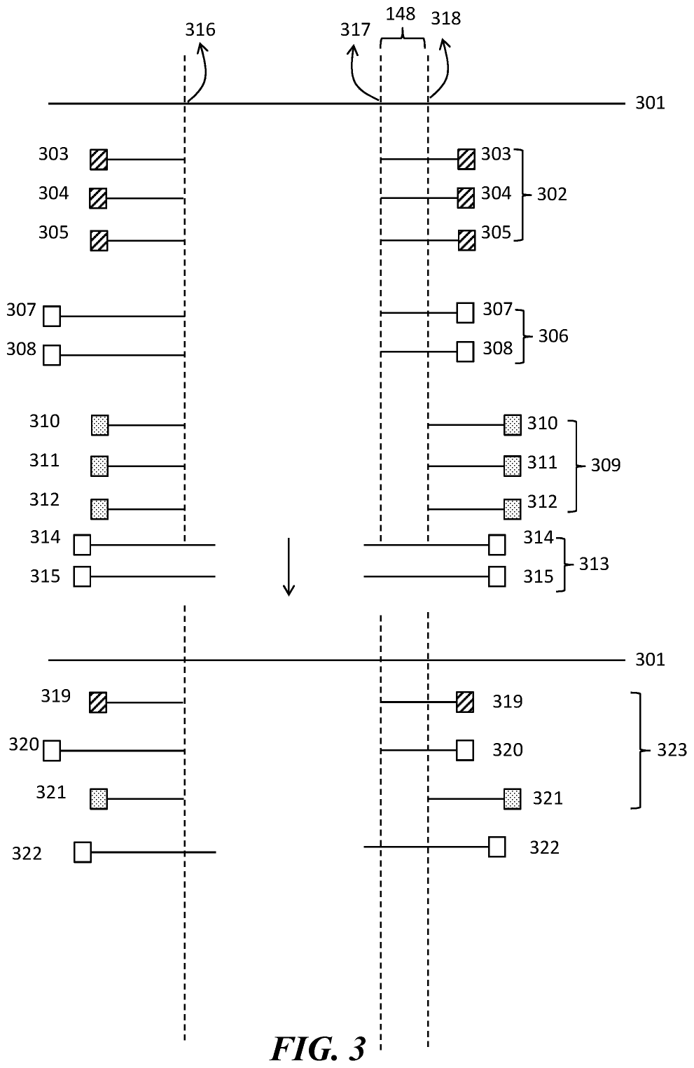

For example, as shown in FIG. 3, split reads within a family are mapped to a reference sequence 301. A first family 302 comprises a first set of split reads 303, 304 and 305. A second family 306 comprises a second set of split reads 307 and 308. A third family 309 comprises a third set of split reads 310, 311 and 312. A fourth family 313 comprises a fourth set of split reads 314 and 315.

The first set of split reads and the second set of split reads map to genetic loci adjacent to a first breakpoint pair 316 and 317. The third set of split reads map to genetic loci adjacent a second breakpoint pair 316 and 318. The fourth set of split reads do not map to any genetic loci adjacent to the breakpoints 316, 317 or 318.

In some embodiments, split read consensus sequences from families may cluster around a breakpoint pair and may form a fusion cluster. For example, the first family 302 is represented by a first split read consensus sequence 319. The second family 306 is represented by a second split read consensus sequence 320. The third family 309 is represented by a third split read consensus sequence 321. The fourth family 313 is represented by a fourth split read consensus sequence 322. The first family 302, the second family 306 and the third family 309 cluster around the breakpoint pairs while the fourth family 313 does not.

In some embodiments, a fusion cluster is detected based on mapping of consensus sequences on the breakpoint pairs. For example, as in FIG. 3, the first split read consensus sequence 319, the second split read consensus sequence 320 and the third split read consensus sequence 321 form a fusion cluster 323. However, the fourth split read consensus sequence 322 is not included in the fusion cluster 323. These split read consensus sequences are included in the fusion cluster in this embodiment because the distance between the respective breakpoints 148 is less than a predetermined breakpoint distance e.g., less than 10 nucleotides. Consensus breakpoints can be called based on, for example, the majority breakpoint in the fusion clusters (breakpoints 316 and 317 in FIG. 3).

In other embodiments, families comprising split reads having similar breakpoint pairs may be grouped into fusion clusters. For example, as in FIG. 3, first family 302, second family 306 and third family 309 cluster around similar breakpoint pairs. These families are included in the fusion cluster in this embodiment because the distance between the respective breakpoints 148 is less than a predetermined breakpoint distance e.g., less than 10 nucleotides. Consensus breakpoints can be called based on, for example, the majority breakpoint in the fusion clusters.

Once the consensus breakpoint pair is identified, genetic variants, such as an insertion, deletion or fusion can be detected.

Distinguishing insertions and deletions (indels) from gene fusions can be performed using an algorithm, e.g., executed by computer. The algorithm can take into consideration one or more factors including, but not limited to: (1) distance between the breakpoint pairs, (2) location of the breakpoints on the same chromosomes, (3) subsequences in the same or different orientation, and/or (4) subsequences in normal or reversed genomic order. If the breakpoints occur on different chromosomes, the variant would always be regarded as a fusion. If the breakpoints are on the same chromosome, but the sub-sequences are in different (opposing) 5′-3′ orientation, the variant would also be regarded as fusion, or in some cases, an inversion. If the breakpoints are on the same chromosome and the subsequences are in the same 5′-3′ orientation, the variant can be called an insertion or deletion if the distance between breakpoint pairs is less than a predetermined maximum distance (e.g., within a gene, less than 5,000 nucleotides, less than 4,000 nucleotides, less than 3,000 nucleotides, less than 2,000 nucleotides, or less than 1,000 nucleotides), otherwise it would be called as a fusion. The insertions and deletions determined using the above criteria can be further distinguished from each other based on whether the sub-sequences are in normal genomic order (i.e., if the normal order of the subsequences on a chromosome is A-B, then, the order in the target molecules is also A-B—in such case call deletion) or in reversed genomic order (i.e., if the normal order of the subsequences on a chromosome is A-B, then, the order in the target molecules is B-A—in such case call insertion). If the above rule established a deletion, the actual deleted sequence is between the two breakpoints. If the above rule established an insertion, a copy of the sequence between the two breakpoints is inserted next to one of the breakpoints (i.e., the sequence between the two breakpoints is duplicated). The sub-sequences may refer to the sequence of a split read within the families or a sequence of a family consensus sequence.

In some embodiments, the predetermined maximum distance between breakpoint pairs may be less than 5,000 nucleotides, less than 4,500 nucleotides, less than 4,000 nucleotides, less than 3,500 nucleotides, less than 3,000 nucleotides, less than 2,500 nucleotides, less than 2,000 nucleotides, less than 1,500 nucleotides, less than 1,000 nucleotides, less than 500 nucleotides, or less than 250 nucleotides. In some embodiments, the predetermined maximum distance between breakpoint pairs is less than the number of nucleotides of a region within a target gene of interest (e.g., less than the length of exon 14 in MET).

In certain embodiments, systems and methods disclosed herein are particularly useful for detecting midsize indels (such as those between 21-50 nucleotides, for example) and/or long indels (such as those greater than 50 nucleotides, greater than 100 nucleotides, greater than 500 nucleotides, greater than 1,000 nucleotides, greater than 2,000 nucleotides, greater than 3,000 nucleotides, greater than 4,000 nucleotides, greater than 5,000 nucleotides, greater than 10,000 nucleotides, an entire exon and/or intron, or an entire gene, for example).

In some embodiments, the insertion and/or deletion may occur within genes that include, but are not to be limited to, the group consisting of APC, ARID1A, ARID1B, ATM, BRCA1, BRCA2, CDH1, CDKN2A, EGFR, ERBB2, FMN2, GATA3, KIT, MET, MECP2, MLH1, MTOR, NF1, PDGFRA, PGAP3, PRODH, PTEN, RB1, SMAD4, SRD5A3, STK11, TP53, TSC1, VHL, and UBE3A. In some embodiments, the insertion and/or deletion may occur within genes that include, but are not to be limited to, EGFR (exons 18-21), ERBB2 (exons 19 and 20), ESR1 (exon 10), MET (exons 13-14 and intron 13-14), BRAF (exon 15), CTNNB1 (exon 3), FGFR2 (exon 6), GATA2 (exons 5-6), GNAS (exon 8), IDH1 (exon 4), IDH2 (exon 4), KIT (exons 1-21), KRAS (exons 2-3), NRAS (exons 2-3), PIK3CA (exon 10 and 21), PTEN (exon 5), SMAD4 (exon 12), TP53 (exons 4-8 and 11). In certain embodiments, the insertion and/or deletion may include, but not be limited to, a frameshift mutation, a non-frameshift mutation, an inversion (chromosomal rearrangement), whole exon deletions, and/or a tandem duplication.

In some embodiments, a fusion can be called when family consensus sequences comprised in a fusion cluster fail to meet any or all of the criteria for calling an insertion and/or deletion.

An algorithm for calling an insertion and/or deletion and/or fusion may include mapping processed reads to a reference sequence and assigning a unique read identifier to the processed read. Based on the alignment of the processed reads, breakpoints and breakpoint pairs are determined on the reference sequence to determine the processed reads having fusions. The breakpoints and the breakpoint pairs may be reported by breakpoint IDs and the number of the processed reads aligned to the breakpoints and breakpoint pairs. The processed reads having similar breakpoints are grouped into families based on common breakpoint pairs. The reads of families, or consensus sequences of the families, are then grouped into a fusion cluster based on breakpoints within a predetermined breakpoint distance of each other. The predetermined breakpoint distance between the breakpoints in the reference sequence may be less than 25 nucleotides or less than 10 nucleotides or 5 nucleotides.

The processed reads with a fusion cannot be mapped contiguously to the reference sequence. The breakpoints in the processed read with a fusion can include a mapped portion and a clipped portion that cannot be mapped contiguously to the reference sequence. A fusion is called when the processed reads map to at least two breakpoints and map to the same strand (e.g. 5′ strand or 3′ strand). Fusion in the processed read can be determined using a voting method, in which the breakpoint among all the breakpoints having the most aligned processed reads is called a fusion breakpoint. The breakpoints of different processed reads may be weighted using a quality algorithm.

In some embodiments, the fusions detected may be associated with genes that include, but are not to be limited to, the group consisting of ALK, FGFR2, FGFR3, TRK1, RET, and/or ROS1.

The systems and methods may be particularly useful in the analysis of cell free DNAs. Cell free DNA may be extracted from any number of subjects, such as subjects without cancer, subjects at risk for cancer, or subjects known to have cancer (e.g. through other means).

In some embodiments, the methods of the present disclosure may include a step of generating a report in electronic format, which provides an indication of polynucleotide molecules having or not having the insertions and/or deletions and/or fusions.

The term “polynucleotide” or “polynucleotide sequence” or “polynucleotide molecule,” as used herein, generally refers to a molecule comprising one or more nucleic acid subunits. A polynucleotide can include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a polynucleotide is deoxyribonucleic acid (DNA) or ribonucleic acid (RNA), or derivatives thereof. A polynucleotide can be single-stranded or double stranded.

Polynucleotides can comprise sequences associated with cancer. The cancer-associated sequences can comprise single nucleotide variation (SNV), copy number variation (CNV), insertions, deletions, and/or rearrangements.

The term “subject,” as used herein, generally refers to an animal, such as a mammalian species (e.g., human) or avian (e.g., bird) species, or other organism, such as a plant. More specifically, the subject can be a vertebrate, a mammal, a mouse, a primate, a simian or a human. Animals include, but are not limited to, farm animals, sport animals, and pets. A subject can be a healthy individual, an individual that has or is suspected of having a disease or a pre-disposition to the disease, or an individual that is in need of therapy or suspected of needing therapy. A subject can be a patient.

Sequencing methods may include, but are not limited to: Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS)(Helicos), massively-parallel sequencing, Clonal Single Molecule Array (Solexa), shotgun sequencing, Maxim-Gilbert sequencing, primer walking, sequencing using PacBio, SOLiD, Ion Torrent, or Nanopore platforms and any other sequencing methods known in the art.

After sequencing data of cell free DNA sequences are collected as sequencing reads, one or more bioinformatics processes may be applied to the sequencing reads. Additional bioinformatics processes may be simultaneously or subsequently applied to detect genetic features or aberrations such as copy number variation, rare mutations (e.g., single or multiple nucleotide variations) or changes in epigenetic markers, including but not limited to methylation profiles.

A variety of different reactions and/operations may occur within the systems and methods disclosed herein, including but not limited to: nucleic acid sequencing, nucleic acid quantification, sequencing optimization, detecting gene expression, quantifying gene expression, genomic profiling, cancer profiling, or analysis of expressed markers. Moreover, the systems and methods have numerous medical applications. For example, it may be used for the identification, detection, diagnosis, treatment, staging of, or risk prediction of various genetic and non-genetic diseases and disorders including cancer. It may be used to assess subject response to different treatments of the genetic and non-genetic diseases, or provide information regarding disease progression and prognosis.

Accordingly, all embodiments of the disclosure can be implements as methods for determining genetic variants, including insertions and/or deletions and/or fusions. In some embodiments, these genetic can be used for the identification, detection, diagnosis, treatment, staging of, or risk prediction of various genetic and non-genetic diseases. In some embodiments, the disease is cancer.

Computer Systems

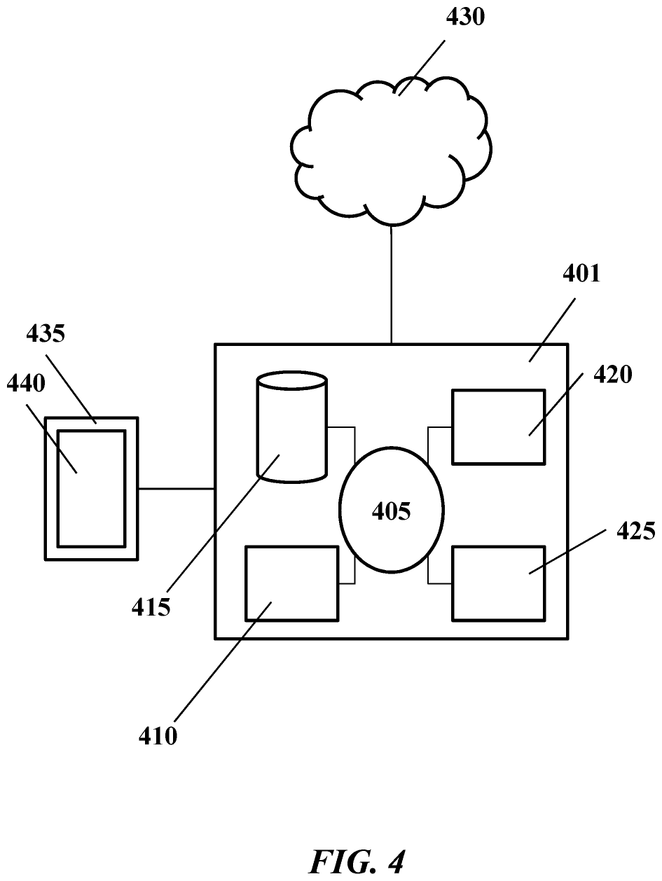

Methods of the present disclosure can be implemented using, or with the aid of, computer systems. For example, the methods of (i) merging the overlapping regions of paired-end sequence reads to generate unique sequences, (ii) mapping the unique sequence reads to a reference sequences, (iii) grouping unique sequence reads into families, (iv) grouping unique sequence reads of families into fusion clusters, and/or (v) calling fusion clusters as comprising an insertion and/or deletion and/or fusions, can be performed with a computer processor. FIG. 4 shows a computer system 401 that is programmed or otherwise configured to implement the methods of the present disclosure. The computer system 401 can regulate various aspects sample preparation, sequencing and/or analysis. In some examples, the computer system 401 is configured to perform sample preparation and sample analysis, including nucleic acid sequencing.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication network or bus (solid lines), such as a motherboard. The storage unit 415 can be a data storage unit (or data repository) for storing data. The computer system 401 can be operatively coupled to a computer network 430 with the aid of the communication interface 420. The computer network 430 can be the Internet, an internet and/or extranet, or an intranet and/or extranet that is in communication with the Internet. The computer network 430 in some cases is a telecommunication and/or data network. The computer network 430 can include one or more computer servers, which can enable distributed computing, such as cloud computing. The computer network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. Examples of operations performed by the CPU 405 can include fetch, decode, execute, and writeback.

The storage unit 415 can store files, such as drivers, libraries and saved programs. The storage unit 415 can store programs generated by users and recorded sessions, as well as output(s) associated with the programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display that comprises a user interface (UI) for providing, for example, one or more results of sample analysis. Examples of UI's include, without limitation, a graphical user interface (GUI) and web-based user interface.

Applications

A. Early Detection of Cancer

Numerous cancers may be detected using the methods and systems described herein.

Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancers individuals using the methods and systems described herein.

For example, blood from subjects at risk for cancer may be drawn and prepared as described herein to generate a population of cell free polynucleotides. In one example, this might be cell free DNA. The systems and methods of the disclosure may be employed to detect rare mutations or copy number variations that may exist in certain cancers present. The method may help detect the presence of cancerous cells in the body, despite the absence of symptoms or other hallmarks of disease.

The types and number of cancers that may be detected may include but are not limited to blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogeneous tumors and the like.

In the early detection of cancers, any of the systems or methods herein described, including rare mutation detection or copy number variation detection may be utilized to detect cancers. These system and methods may be used to detect any number of genetic aberrations that may cause or result from cancers. These may include but are not limited to mutations, rare mutations, indels, copy number variations, transversions, translocations, inversion, deletions, chromosomal instability, chromosomal structure alterations, gene fusions, chromosome fusions, gene truncations, gene amplification, gene duplications, chromosomal lesions, DNA lesions, and cancer.

Additionally, the systems and methods described herein may also be used to help characterize certain cancers. Genetic data produced from the system and methods of this disclosure may allow practitioners to help better characterize a specific form of cancer. Often times, cancers are heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer.

B. Cancer Treatment, Monitoring and Prognosis

The systems and methods provided herein may be used to treat or monitor already known cancers, or other diseases in a particular subject. This may allow either a subject or practitioner to adapt treatment options in accord with the progress of the disease. In this example, the systems and methods described herein may be used to construct genetic profiles of a particular subject of the course of the disease. In some instances, cancers can progress, becoming more aggressive and genetically unstable. In other examples, cancers may remain benign, inactive, dormant or in remission. The system and methods of this disclosure may be useful in determining disease progression, remission or recurrence.

Further, the systems and methods described herein may be useful in determining the efficacy of a particular treatment option. In one example, successful treatment options may actually increase the amount of indels detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the systems and methods described herein may be useful in monitoring residual disease or recurrence of disease.

C. Early Detection and Monitoring of Other Diseases or Disease States

The methods and systems described herein may not be limited to detection of indels associated with only cancers. Various other diseases and infections may result in other types of conditions that may be suitable for early detection and monitoring. For example, in certain cases, genetic disorders or infectious diseases may cause a certain genetic mosaicism within a subject. This genetic mosaicism may cause copy number variation and rare mutations that could be observed

Further, the systems and methods of this disclosure may also be used to monitor systemic infections themselves, as may be caused by a pathogen such as a bacteria or virus. Indel detection may be used to determine how a population of pathogens is changing during the course of infection. This may be particularly important during chronic infections, such as HIV/AIDS or Hepatitis infections, whereby viruses may change life cycle state and/or mutate into more virulent forms during the course of infection.

Further, the methods of the disclosure may be used to characterize the heterogeneity of an abnormal condition in a subject, the method comprising generating a genetic profile of extracellular polynucleotides in the subject, wherein the genetic profile comprises a plurality of data resulting from indel analyses. In some cases, including but not limited to cancer, a disease may be heterogeneous. Disease cells may not be identical. In the example of cancer, some tumors are known to comprise different types of tumor cells, some cells in different stages of the cancer. In other examples, heterogeneity may comprise multiple foci of disease. Again, in the example of cancer, there may be multiple tumor foci, perhaps where one or more foci are the result of metastases that have spread from a primary site.

The methods of this disclosure may be used to generate or profile, fingerprint or set of data that is a summation of genetic information derived from different cells in a heterogeneous disease. This set of data may comprise copy number variation and rare mutation analyses alone or in combination.

D. Early Detection and Monitoring of Other Diseases or Disease States of Fetal Origin

Additionally, the systems and methods of the disclosure may be used to diagnose, prognose, monitor or observe cancers or other diseases of fetal origin. That is, these methodologies may be employed in a pregnant subject to diagnose, prognose, monitor or observe cancers or other diseases in a unborn subject whose DNA and other polynucleotides may co-circulate with maternal molecules.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Examples

### Example 1: Detecting MET Exon 14 Skipping Deletions from 27 Different Samples

A set of patient samples was processed and analyzed using a blood-based DNA assay developed by Guardant Health, Inc. (Redwood City, CA). The sequence reads were analyzed for genetic variants. As shown in Table 1 below, 27 different samples among the set were detected to have fusion clusters.

TABLE 1

| | | | Distance |
|---|---|---|---|
| Chromosome | Breakpoint 1 | Breakpoint 2 | between the |
| Number | Position | Position | Breakpoint Pair |
| 7 | 116411784 | 116412936 | 1152 |
| 7 | 116411846 | 116411988 | 142 |
| 7 | 116411947 | 116412086 | 139 |
| 7 | 116411764 | 116412001 | 237 |
| 7 | 116411750 | 116411971 | 221 |
| 7 | 116411763 | 116411986 | 223 |
| 7 | 116411794 | 116412002 | 208 |
| 7 | 116411808 | 116411918 | 110 |
| 7 | 116411765 | 116411966 | 201 |
| 7 | 116411861 | 116412289 | 428 |
| 7 | 116411757 | 116411959 | 202 |
| 7 | 116411810 | 116412011 | 201 |
| 7 | 116411845 | 116412479 | 634 |
| 7 | 116411825 | 116411924 | 99 |
| 7 | 116411754 | 116411965 | 211 |
| 7 | 116411711 | 116411913 | 202 |
| 7 | 116411927 | 116412165 | 238 |
| 7 | 116411730 | 116412426 | 696 |
| 7 | 116411807 | 116411915 | 108 |
| 7 | 116411795 | 116412053 | 258 |
| 7 | 116411966 | 116412065 | 99 |
| 7 | 116411919 | 116412847 | 928 |
| 7 | 116411755 | 116411971 | 216 |
| 7 | 116411749 | 116411981 | 232 |
| 7 | 116412001 | 116412336 | 335 |
| 7 | 116412011 | 116412221 | 210 |
| 7 | 116411741 | 116411963 | 222 |

In Table 1, each row represents a fusion cluster with a consensus breakpoint pair. The fusion clusters met the criteria for calling a deletion, including (1) breakpoint pairs mapping to the same chromosome-chromosome 7, (2) the sub-sequences were found to be in the same 5′-3′ orientation, and (3), the distance between breakpoint positions 1 and 2 were within the predetermined maximum distance—in this case, 3,222 nucleotides, and additionally, (4) are in normal genomic order as compared to a reference sequence. Reference alignment of the sequence reads indicated that the detected genetic variant was a MET exon 14 skipping deletion.

What is claimed is:

1. A method for treating a subject having a cancer characterized at least by a MET exon 14 skipping deletion, comprising:
(a) obtaining or having obtained genetic results from a sample of a subject, wherein the genetic results comprise an indication of a presence of the MET exon 14 skipping deletion in the sample of the subject, wherein the genetic results are generated by a method comprising:
(1) receiving genetic sequence reads generated by a nucleic acid sequencer, wherein the genetic sequence reads comprise multiple paired-end sequences of polynucleotides;
(2) merging at least a subset of paired-end sequence reads having overlapping regions to produce merged reads;
(3) mapping the merged reads to a reference sequence, thereby generating mapped merged reads;
(4) grouping the mapped merged reads into families based at least on sequence information at start base positions or stop base positions of the mapped merged reads, wherein a family from among the families corresponds to the cell-free nucleic acid molecule from among the cell-free nucleic acid molecules of the sample, wherein grouping the mapped merged reads into the families further comprises compacting a portion of a mapped merged read from among the mapped merged reads to remove duplicate nucleotides in a homopolymer, thereby reducing a number of the families produced because of sequencing errors in the genetic sequence reads;
(5) grouping at least a portion of the families into the fusion clusters, each of the fusion clusters comprising a plurality of split reads, wherein a split read among the plurality of split reads comprises a first sub-sequence adjacent to a first breakpoint that maps to a first genetic locus of the reference sequence and a second sub-sequence adjacent to a second breakpoint that maps to a second genetic locus of the reference sequence different from the first genetic locus, and wherein the first breakpoint and the second breakpoint form a breakpoint pair;
(6) detecting the presence of the MET exon 14 skipping deletion in a fusion cluster from among the fusion clusters when:
A) breakpoint pairs from among the plurality of split reads in the fusion cluster map to the same chromosome;
B) a distance between the first breakpoint and the second breakpoint in the breakpoint pair is less than a predetermined distance on the reference sequence;
C) the first and second sub-sequences are in a same 5′-3′ orientation; and
(b) administering a MET inhibitor to the subject to treat the cancer based on detecting the presence of the MET exon 14 skipping deletion, wherein the MET inhibitor is selected from the group consisting of crizotinib, cabozantinib, capmatinib, tepotinib, and glesatinib.

2. The method of claim 1, wherein the sample comprises between 1 nanogram and 500 nanograms of cell-free nucleic acid molecules from the subject.

3. The method of claim 1, wherein the genetic sequence reads are derived from the cell-free nucleic acid molecules or derivatives thereof.

4. The method of claim 1, wherein the method comprises detecting a deletion when the first and second sub-sequences are in normal genomic order as compared to the reference sequence.

5. The method of claim 1, wherein the method comprises detecting an insertion when the first and second sub-sequences are in reverse genomic order as compared to the reference sequence.

6. The method of claim 1, wherein the method comprises merging the paired-end sequence reads with an overlapping region having at least 70% identity.

7. The method of claim 1, wherein the method comprises merging the paired-end sequence reads with an overlapping region of at least 13 bases.

8. The method of claim 1, wherein the method comprises processing merged reads to generate processed reads comprising representative, merged unique reads.

9. The method of claim 1, wherein the multiple paired-end sequences of the polynucleotides comprise molecular barcoding sequence information.

10. The method of claim 1, wherein the method comprises generating a consensus sequence for each family of the at least the portion of the families.

11. The method of claim 1, wherein the distance between the first breakpoints of the plurality of split reads within the fusion cluster is than 10 nucleotides, and wherein a distance between the second breakpoints of the plurality of split reads within the fusion cluster is less than 10 nucleotides.

12. The method of claim 1, wherein the predetermined distance is less than 5,000 nucleotides.

13. The method of claim 1, wherein the families comprise mapped merged reads:
having a same start position and a same compacted stop sequence, or
having a same stop position and a same compacted start sequence.

14. The method of claim 1, wherein the homopolymer comprises a poly(dA) or a poly(dT).

15. The method of claim 1, wherein the homopolymer comprises a poly(dG) or a poly(dC).

16. The method of claim 1, wherein the method comprises assessing a quality of the paired-end sequence reads to generate quality scores.

17. The method of claim 1, wherein the predetermined distance is less than 4,000 nucleotides.

18. The method of claim 1, wherein the sample comprises different types of tumor cells.

19. The method of claim 1, wherein the type of cancer comprises: blood cancer, brain cancer, lung cancer, skin cancer, nose cancer, throat cancer, liver cancer, bone cancer, a type of lymphoma, pancreatic cancer, skin cancer, bowel cancer, rectal cancer, thyroid cancer, bladder cancer, kidney cancer, mouth cancer, stomach cancer, solid state tumor, heterogeneous tumor, or homogeneous tumor.

20. The method of claim 1, wherein the method comprises:
providing feedback about progression of the type of cancer; or
specifying aggressiveness and genetic stability of the type of cancer.
